(19) United States
(12) Patent Application Publication
(10) Pub. No.: US 2003/0170377 A1
(43) Pub. Date: Sep. 11, 2003

(54) Method for minimizing high altitude pulmonary edema

(76) Inventor: Harold T. Hammel, Ellettsville, IN (US)

Correspondence Address: Robert J. Yarbrough, Attorney at Law, 201 North Jackson St., Media, PA, 19063, US

(21) Appl. No.: 10/090,649

(22) Filed: Mar. 5, 2002

(51) Int. Cl. A61K 47/00; A23G 3/00

(52) U.S. Cl. 426/658; 424/439

(57) ABSTRACT

The invention comprises a method for reducing the incidence of High Altitude Pulmonary Edema (“HAPE”) based on a valid understanding of the process of osmosis. Diffusion of bicarbonate ions through alveolar capillaries drags upon the water through which the ions diffuse in the same manner as if a reduced pressure were applied to the water. The resulting osmotic effect present in the capillary as a result of the bicarbonate diffusion draws edemateous fluid from the alveoli into the capillary. HAPE can be minimized through adjusting the diet to maximize bicarbonate ions in the plasma and hence to increase diffusion and the resulting osmotic effect.

### BACKGROUND OF THE INVENTION

1. Field of the Invention

The invention is a method for minimizing the disorder of high altitude pulmonary edema (“HAPE”) based on a correct understanding of the process of osmosis.

2. Description of the Related Art

A correct understanding of the process of osmosis makes clear the mechanism of the flow of blood plasma out of and into alveolar capillaries and reveals one of the contributing causes of High Altitude Pulmonary Edema (“HAPE”). The same correct understanding of osmosis makes clear many biological processes, such as intra-ocular pressure and removal of aqueous humor from the anterior chamber of the eye.

HAPE is a severe disorder experienced by persons exposed to low atmospheric pressure, principally mountain climbers at high altitudes. HAPE is characterized by extreme fatigue, breathlessness at rest, a cough that may produce frothy or pink sputum, gurgling or rattling sounds during breathing, chest tightness, fullness or congestion, and blue or gray lips or fingernails. Unless treated, HAPE can progress to coma and death.

### SUMMARY OF THE INVENTION

An understanding of the process of osmosis reveals one of the contributing causes of HAPE. In a healthy individual at sea level, hydrostatic pressure in the pulmonary capillaries forces, or extravasates, fluid continuously through the walls of the pulmonary capillaries into the alveoli. If a mechanism did not exist to remove the extravasated fluid from the alveoli continuously, healthy persons at sea level would experience pulmonary edema.

In a healthy person at sea level, extravasated fluid is continuously removed from the alveoli to the pulmonary capillaries by the osmotic effect of bicarbonate (HCO₃⁻) ions diffusing within plasma from the arterial end toward the venous end of the capillaries. The diffusion of these bicarbonate (HCO₃⁻) ions within the capillary plasma drags on the plasma water through which the HCO₃⁻ ions diffuse. The plasma water is altered like pure liquid water is altered by lowering the pressure applied to the pure liquid water in an amount equal to the osmotic effect of the diffusing bicarbonate (HCO₃⁻) ions. The altered plasma water pulls fluid from the alveoli of the lungs and into the plasma of the capillaries continuously and thereby prevents pulmonary edema. The rate fluid is removed from the alveoli is proportional to the metabolic rate; i.e., the rate HCO₃⁻ ions are produced by oxidation of carbon in foodstuffs.

In the hypoxic environment of the mountain climber at high altitude, too little oxygen is available for metabolism of carbon. Too little carbon is oxidized to CO2 and too little bicarbonate (HCO₃⁻) is carried as a waste product of metabolism in the plasma flowing to the pulmonary capillaries. Because the concentration of HCO₃⁻ ions in the capillaries is reduced, there is insufficient diffusion of bicarbonate ions from the arterial to the venous end within the pulmonary capillaries. As a result, the osmotic effect is reduced and insufficient fluid is removed from the pulmonary alveoli. The buildup of edemateous fluid in the mountain climber contributes to the symptoms of HAPE.

The occurrence of HAPE can be minimized through adjustment of the diet of the mountain climber. The effects of HAPE can be minimized by (1) eliminating all nitrogen-bearing foodstuffs such as meat and legumes from the diet, (2) maximizing the carbon content and the oxygen content of the diet, and (3) minimizing the hydrogen content of the diet. The high altitude diet should maximize the production of carbon dioxide and also require the least amount of inspired oxygen to metabolize ingested carbon and hydrogen and to metabolize nitrogen from tissue. A diet of pure glucose (C₆H₁₂O₆) and/or sucrose (C₁₂H₂₄O₁₂) is recommended. Glucose has sufficient oxygen to metabolize half its carbon or all of its hydrogen. Metabolism of the remaining carbon or hydrogen requires inspired oxygen.

### BRIEF DESCRIPTION OF THE DRAWINGS

FIG. 1—Schematic diagram of an alveoli and capillary of a healthy individual at sea level

FIG. 2—Schematic diagram of an alveoli and capillary subject to HAFE

FIG. 3—Schematic diagram illustrating the water concentration of pure water prior to the addition of NaF or MgSO4 to the water.

FIG. 4—Schematic diagram showing the increase in the water concentration in a solution after addition of NaF or MgSO4. Note that the increase in water concentration is exaggerated for the sake of illustration.

FIG. 5—Schematic diagram illustrating that the water concentration theory of osmosis is invalid because it does not correctly predict the osmotic effect of a solution of NaF or MgSO4.

FIG. 6—Schematic diagram illustrating the increase in concentration of HCO₃⁻ ions in plasma in systemic tissue.

FIG. 7—Schematic diagram illustrating the diffusion of HCO₃⁻ in systemic tissue from the area of high concentration to the area of low concentration against the direction of plasma flow. Note that the representation of relative numbers of HCO₃⁻ ions is exaggerated for purposes of illustration.

FIG. 8—Schematic diagram illustrating the decrease in concentration of HCO₃⁻ in plasma in alveolar tissue. Note that the representation of relative numbers of HCO₃⁻ ions is exaggerated for purposes of illustration.

FIG. 9—Schematic diagram illustrating the diffusion of HCO3—in alveolar tissue from the area of high concentration to the area of low concentration in the direction of plasma flow. Note that the representation of relative numbers of HCO₃⁻ ions is exaggerated for purposes of illustration.

FIG. 10—Flow diagram illustrating steps to minimize HAPE

FIG. 11—Second flow diagram illustrating steps to minimize HAPE

### DESCRIPTION OF THE PREFERRED EMBODIMENT

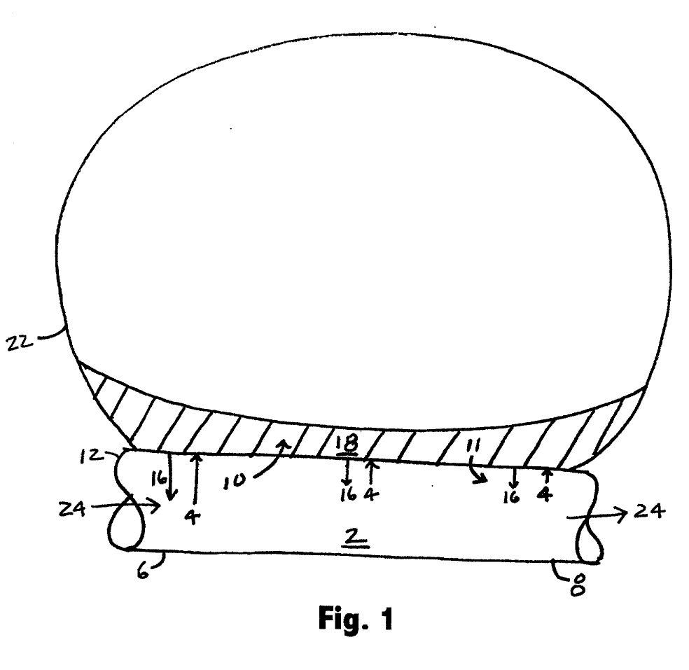

The heart maintains the entire circulatory system at an elevated hydrostatic pressure. As shown in FIG. 1, the hydrostatic pressure 4 in a capillary 2 is greatest on the arterial end 6 of the capillary 2 and less on the venous end 8, but always is a positive pressure. In the portion of the capillary 2 near to the arterial end 6, the hydrostatic pressure 4 forces, or “extravasates,” fluid 10 through the walls 12 of the capillary 2 into surrounding tissue, which may be an alveolus 22 in the lung (FIG. 1). An alveolus of a healthy person normally contains some interstitial fluid 18. The hydrostatic pressure 4 is countered by osmotic effect 16 that returns return fluid 11 from interstitial fluid 18 to the venous end 6 of the capillary 2.

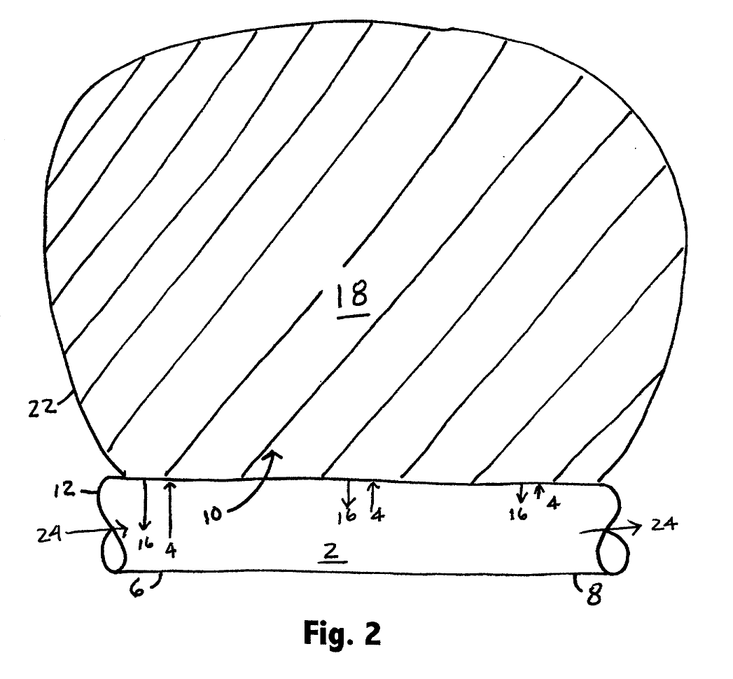

In pulmonary edema (FIG. 2), the osmotic effect 16 is not great enough to overcome the hydrostatic pressure 4 in the capillary 2 and contributes to the accumulation of extravasated fluid 18 in the alveoli 22 of the lung.

Physiologists acknowledge that they do not fully understand the cause of high altitude pulmonary edema^{1,2}. This is not surprising because they have accepted uncritically Starling's hypothesis^{3,4,5} as the basis for understanding the exchange of fluid 10 between plasma 24 in a capillary 2 and the interstitial fluid 18 surrounding the capillary 2 and in the alveolus 22. The acceptance of Starling's hypothesis by physiologists is based on the Lewis theory, an unrealistic interpretation of the nature of osmosis. Most physical chemists and chemical thermodynamicists also do not understand how solute lowers the chemical potential of water in an aqueous solution.^{6,7,8}

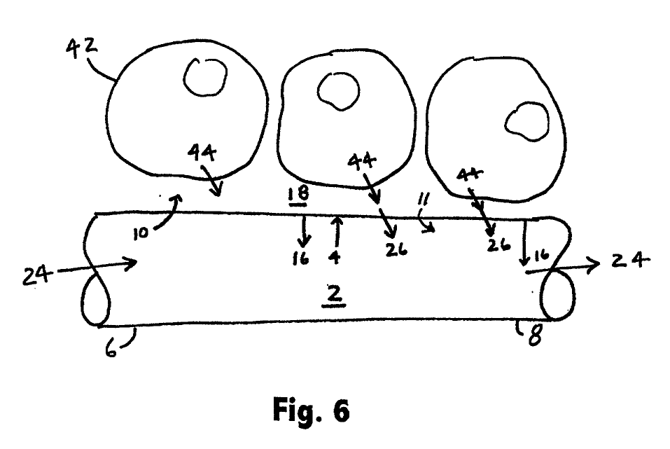

The following discussion demonstrates that the Lewis theory of osmosis and Starling's hypothesis are incorrect. As discussed below, the Hulett theory correctly describes the process of osmosis. Contrary to the Starling hypothesis, the osmotic force 16 returning interstitial fluid 18 to capillaries 2 is caused mainly by diffusion within the capillary of HCO₃⁻ ions 26 (FIGS. 6-9).

A. Nature of Osmosis

1. Lewis's Incorrect Theory of Osmosis

Chemists have accepted G. N. Lewis's unrealistic account of osmosis even though it does not explain how solvent in a solution lowers the chemical potential of the solvent in a solution. In 1908, Lewis⁹ proposed that

$${n}_{solute}^{1}$$

moles of solute lowers the “activity” of water,

$${a}_{H2O}^{1} ( T , {P}_{e}^{1} , {n}_{solute}^{1} , {n}_{H2O}^{1} ) ,$$

when dissolved in

$${n}_{H2O}^{1}$$

moles of water in an aqueous solution at a temperature (T) and external pressure (pₑ¹) applied to the solution. He then proposed that this lowering of the “activity” of the water causes the “chemical potential” of the water in the solution,

$${μ}_{H2O}^{1} ( T , {p}_{e}^{1} , {n}_{solute}^{1} , {n}_{H2O}^{1} ) ,$$

to be less than

$${μ}_{H2O}^{1 *} ( T , {p}_{e}^{1} ) ,$$

the chemical potential of pure liquid water at the same applied temperature and pressure (T,pₑ¹). Lewis proposed the relationship between activity and chemical potential of the water in the solution to be:

$${μ}_{H2O}^{1} ( T , {p}_{e}^{1} , {n}_{solute}^{1} , {n}_{H2O}^{1} ) - {μ}_{H2O}^{1 *} ( T , {p}_{e}^{1} ) ≡ R T ln {a}_{H2O}^{1} ( T , {p}_{e}^{1} , {n}_{solute}^{1} , {n}_{H2O}^{1} ) ,$$

where R is the universal gas constant and T is the absolute temperature. A widely accepted implication is that solute lowers the activity of the water in the solution by lowering the “Fugacity” of the water and that this explains why the chemical potential of the water is lessened an amount stated by this thermodynamic equation. As a matter of fact, this equation is nothing more than a definition of the term “activity of water in the solution”, as indicated by≡ between the two sides. It does not explain how the solute lowers the Fugacity or the activity or the chemical potential of the water in the solution. Water activity is a dimensionless number greater than zero and less than or equal to one. Adding solute does lower the chemical potential of water so that the left side of the defining equation becomes negative. Water activity, by definition, becomes less than one so that 1 n a_{H2O}¹ becomes negative. However, this account does not explain how the solute lowers the chemical potential or the activity of the water in the solution.

Another accepted implication of the Lewis account of osmosis is that the solute acts on the water and lowers its chemical potential at or near the semi-permeable membrane that separates pure liquid water from water in the solution. The presumption is that water molecules diffuse from pure liquid water at a higher chemical potential through the semi-permeable membrane into the solution where the chemical potential of its water is less. Diffusion is said to continue until the rising pressure in the solution water increases the chemical potential of the water in the solution to equal the higher chemical potential of the pure liquid water beyond the semi permeable membrane.

2. Hulett Theory of Osmosis.

Lewis ignored a valid and prior explanation of how solute alters the water in an aqueous solution. In 1903, Hulett^{6,7} correctly concluded that solute alters the internal tension in the force bonding the water molecules together in the liquid phase. When

$${π}_{H2O}^{1} ( T , {p}_{e}^{1} , {n}_{solute}^{1} , {n}_{H2O}^{1} )$$

denotes the osmotic pressure of the water at a distensible boundary of the solution, Hullet recognized that the solute alters every partial molar property of the water in the solution just like the same molar property of pure liquid is altered by reducing the pressure applied to it by

$${π}_{H2O}^{1} ( T , {p}_{e}^{1} , {n}_{solute}^{1} , {n}_{H2O}^{1} ) .$$

Regarding the chemical potential,

$${μ}_{H2O}^{1} ( T , {p}_{e}^{1} , {n}_{solute} , {n}_{H2O} ) = {μ}_{H2O}^{1 *} ( T , {p}_{e}^{1} - {π}_{H2O}^{1} ( T , {p}_{e}^{1} , {n}_{solute}^{1} , {n}_{H2O}^{1} ) ) .$$

Likewise for the other molar properties,

$${p}_{H2O}^{g} ( T , {p}_{e}^{1} , {n}_{solute} , {n}_{H2O} ) = {p}_{H2O}^{g *} ( T , {p}_{e}^{1} - {π}_{H2O}^{1} ( T , {p}_{e}^{1} , {n}_{solute}^{1} , {n}_{H2O}^{1} ) ) ,$$

where

$${p}_{H2O}^{g}$$

is the vapor pressure. And

$$\begin{matrix}{V}_{H2O}^{1} ( T , {p}_{e}^{1} , {n}_{solute} , {n}_{H2O} ) = {V}_{H2O}^{1 *} ( T , {p}_{e}^{1} - {π}_{H2O}^{1} ( T , {p}_{e}^{1} , {n}_{solute}^{1} , {n}_{H2O}^{1} ) ) , \\ {U}_{H2O}^{1} ( T , {p}_{e}^{1} , {n}_{solute} , {n}_{H2O} ) = {U}_{H2O}^{1 *} ( T , {p}_{e}^{1} - {π}_{H2O}^{1} ( T , {p}_{e}^{1} , {n}_{solute}^{1} , {n}_{H2O}^{1} ) ) , \\ {H}_{H2O}^{1} ( T , {p}_{e}^{1} , {n}_{solute} , {n}_{H2O} ) = {H}_{H2O}^{1 *} ( T , {p}_{e}^{1} - {π}_{H2O}^{1} ( T , {p}_{e}^{1} , {n}_{solute}^{1} , {n}_{H2O}^{1} ) ) , \\ {S}_{H2O}^{1} ( T , {p}_{e}^{1} , {n}_{solute} , {n}_{H2O} ) = {S}_{H2O}^{1 *} ( T , {p}_{e}^{1} - {π}_{H2O}^{1} ( T , {p}_{e}^{1} , {n}_{solute}^{1} , {n}_{H2O}^{1} ) ) ,\end{matrix}$$

where

$${V}_{H2O}^{1} , {U}_{H2O}^{1} , {H}_{H2O}^{1} and {S}_{H2O}^{1}$$

are, respectively, the molar volume, molar internal energy, molar enthalpy and molar entropy.

By Hulett's account of osmosis, the altered internal tension of the water in the solution pulls water through the semi-permeable membrane from the pure liquid water until the internal tensions in the water on both sides of the membrane become equal.

3. The Water Concentration Theory, a Modified Lewis Theory, is Disproved by the Properties of Solutions of NaF or MgSO4.

Physiologists continue to reject Hulett's account of osmosis and continue to accept a modified version of Lewis's account^{4,5}. They do so based on a curious application of the process of diffusion. In diffusion, a material in an aqueous solution at a high concentration moves by Brownian motion to an area of lower concentration. Pure liquid water has a concentration of 55.50 moles per liter at 0° C.; that is, the volume occupied by 55.50 moles of pure liquid water is one liter at 0° C. When a solute, say, NaSO4, is added to one liter of pure liquid water, the volume of the resulting NaSO4 solution is greater than the volume of the pure water alone and the concentration of water in the NaSO4 solution falls below 55.50 moles per liter of solution.

Physiologists claim that if the NaSO4 solution is separated from pure water by a semi-permeable membrane, the pure water will diffuse from the water with the higher water concentration (the pure water) to the water with the lower water concentration (the NaSO4 solution). As in Lewis's theory, physiologists presume water molecules diffuse through the membrane until the increasing pressure in the water in the NaSO4 solution raises the chemical potential of the water in the solution to equal the chemical potential of the pure liquid water beyond the membrane.

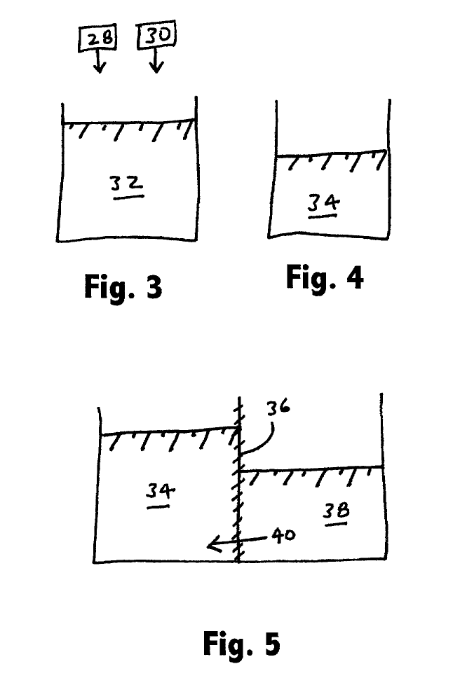

If a single instance is found where the modified Lewis theory does not describe reality, the theory is disproved. As illustrated by FIGS. 3-5, the modified Lewis's theory does not describe reality and hence is proved invalid because the properties of solutions 34 of NaF 28 and MgSO₄ 30 do not conform to the theory. As shown by FIG. 3, if NaF 28 or MgSO₄ 30 is added to pure water 32, the resulting solution 34 (FIG. 4) occupies less volume than did the pure water 32. In other words, when NaF 28 or MgSO₄ 30 is added to water 32, the concentration of water in the solution 34 increases, not decreases. For example, for a NaF 28 solution 34 that is 1000 Osm/Kg water, the water concentration increases to 55.62 mols water per liter of solution at 0° C. 34, an increase of 0.12 mols water per liter of solution at 0° C. 34.

The Physiologist's modified Lewis theory predicts that such a solution would exert a negative osmotic pressure so that water would flow from the solution through a semi-permeable membrane and into adjacent pure water. Contrary to the modified Lewis theory prediction and as shown by FIG. 5, solutions 34 of NaF 28 or MgSO₄ 30 show a positive osmotic pressure 40. Pure water 38 enters a solution 34 of NaF 28 or MgSO₄ 30 through a semi-permeable membrane 36 in about the same amount as would enter a comparable solution of NaSO₄ in which the water concentration in the solution is lower¹⁰.

The physiologist's modification of the Lewis theory is therefore disproved and concentration and/or activity of water in a solution can not provide a mechanism for understanding osmosis. Only Hulett's mechanism provides a valid basis for understanding osmotic effects.

B. Starling's Hypothesis of Fluid Exchange between Plasma and Interstitial Fluid

In 1896, Starling³ performed an experiment in which he concluded that the colloid osmotic pressure of the proteins in plasma exert an osmotic force causing the return of interstitial fluid to the venous end of capillaries. In modern terminology, Starling's hypothesis is expressed as Starling's equation, namely,

$${J}_{v} ( x ) = {L}_{p} ( x ) S ( x ) \{ [ {P}_{i n}^{p1} ( x ) - {P}_{out}^{ISF} ( x ) ] - {σ}_{e} ( x ) [ {COP}_{i n}^{p1} ( x ) - {COP}_{out}^{ISF} ( x ) ] \}$$

According to Starling's hypothesis, four pressures determine whether fluid flows from plasma to interstitial fluid (“ISF”) or from ISF to plasma. Starling³recognized that the hydrostatic pressure in the plasma

$$( {P}_{i n}^{p1} ( x ) )$$

will normally exceed the hydrostatic pressure in ISF

$$( {P}_{out}^{ISF} ( x ) )$$

outside the capillary endothelium along the entire length of the capillary. These differing pressures force the extravasation of fluid into the ISF at the arterial end of the capillary and they comprise the hydrostatic pressure term in the Starling equation. Starling, and subsequent interpreters of Starling's 1896 experiment, postulated another term consisting of the colloid osmotic pressure of plasma

$$( {COP}_{i n}^{p1} ( x ) )$$

and of the colloid osmotic pressure of ISF

$$( {COP}_{out}^{ISF} ( x ) )$$

at the same location along a capillary through which the plasma flows. These two COPs constitute the osmotic force in the Starling equation, where Jᵥ(x) is the volume of fluid filtering through the capillary in unit time and unit length at (x). Lₚ(x) is the hydraulic conductivity of the capillary at (x). S(x) is the circumference of the capillary. σₑ(x) is the reflection coefficient of the endothelium for the colloids.

The second Starling force or osmotic term, i.e.,

$${L}_{p} ( x ) {σ}_{e} ( x ) S ( x ) [ {COP}_{n}^{1} ( x ) - {COP}_{out}^{ISF} ( x ) ] ,$$

states that since the protein concentration in the plasma exceeds the protein concentration in the ISF, this force will return fluid to the capillary when this osmotic force exceeds the hydrostatic force near the venous end of the capillary.

Interpreters of Starling's equation assume that proteins in plasma lower the concentration of water in the plasma. For this reason, they presume that interstitial fluid diffuses into the plasma at the venous end of the capillary where the hydrostatic pressure is least. As noted above, water concentration does not and cannot cause osmotic effects. As Hulett, recognized, the protein molecules in aqueous solution exert a pressure at a distensible boundary of the solution and alter the internal tension of the water in the solution as would lowering the external pressure applied to pure liquid water. The protein molecules also alter the internal tension of the water in the solution and, thereby, lower the chemical potential of the water in the solution. When plasma flows through the capillary at a constant rate (as in Starling's hypothesis), the boundaries of the plasma are already distended by both the colloid pressure and by hydrostatic pressure. That is, when flow is steady, the protein molecules no longer distend the wall of the capillary and have no other effect on fluid exchange between interstitial fluid and plasma.

The purpose of this background review has been: 1) to show that Starling's hypothesis can not be valid and 2) to suggest another force that may be the most important osmotic force in determining the extravasation of fluid from plasma to ISF and in returning most of it from ISF to plasma within the capillary^{10,11,12,13,14}.

C. The Osmotic Force that Accounts for the Return of ISF in Starling's Experiment

1. The Osmotic Force in Systemic Tissue.

a. Changes in Ion Concentration and Electroneutrality.

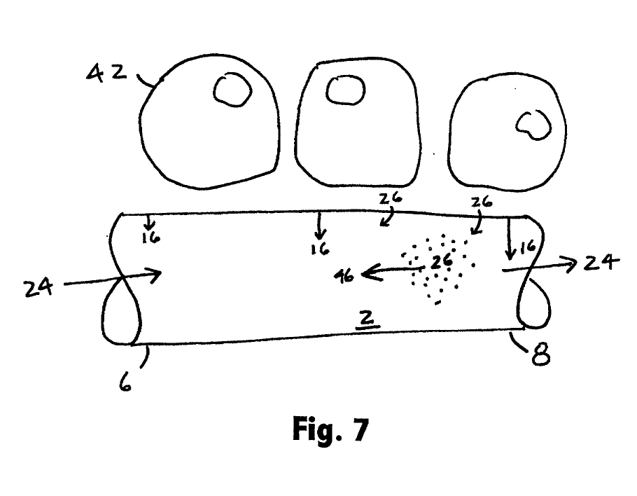

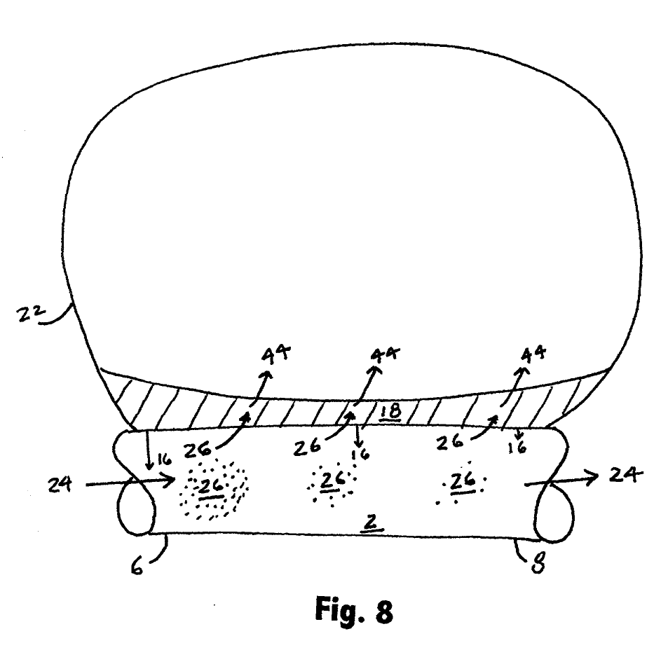

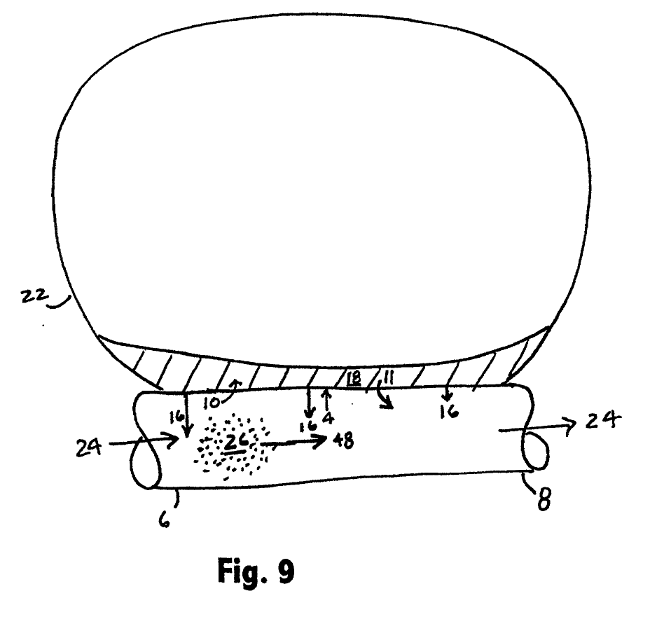

For purposes of this application, “systemic tissue” 42 (FIGS. 6, 7) means all tissue of the body other than the alveolar tissue 22 of the lung (FIGS. 8, 9).

Cells of systemic tissues 42 produce CO2 44 as a waste product of metabolism. The waste CO2 44 diffuses through the interstitial fluid 18 and is carried away in the blood plasma 24 in the form of bicarbonate ions (HCO₃⁻) 26. When plasma 24 flows from the arterial end 6 to the venous end 8 of a capillary 2 in systemic tissue 42, the bicarbonate ion (HCO₃⁻) 26 concentration of the plasma 24 increases from 27.5 to 29 millimols per liter (“mmol/liter”) of plasma in humans at rest¹⁵, an increase in the negative charge of the plasma of 1.5 mmol/liter.

To maintain electrical neutrality, the increase in negative charge of the plasma 24 caused by the increase in HCO₃⁻ ion 26 concentration must be offset by an equivalent increase in the strong ion difference (“SID”) of 1.5 mmol/liter. SID is defined by the equation SID=[Na⁺]−[Cl⁻]. The SID increases as the sodium ion concentration [Na⁺] in the plasma increases and the chloride ion concentration [Cl⁻] decreases as the plasma flows from the arterial end 6 to the venous end 8 of the capillary 2. The chloride ion concentration [Cl⁻] in plasma decreases because the chloride ions enter the red cells in exchange for bicarbonate ions 26. The net effect is to increase the positive charge of the plasma 24 by 1.5 mmol/liter and thereby maintain electroneutrality.

b. Changes in Osmotic Pressure Corresponding to the Changes in Ion Concentration.

The increase in the bicarbonate ion concentration (HCO₃⁻) 26 in the capillary 2 would increase the osmotic effect 16 of the water in plasma 24 by 29 Torr in the absence of other concentration changes. The increase in osmotic effect 16 caused by the increase in concentration of HCO₃⁻ ions 26 is partially offset by a reduction in osmotic pressure caused by changes in the relative concentrations of Na+ and Cl−. The net increase in the osmotic effect 16 of the water in plasma 24 flowing from end to end (6, 8) in a capillary 2 in systemic tissue 42 is about 33 Torr in humans at rest. A Torr is equivalent to the pressure exerted by a column of elemental mercury 1 millimeter tall.

C. Mechanism of the Change in Osmotic Pressure.

To state that a change in concentration of a solute changes the osmotic effect 16 of the water in the plasma 24 says nothing of the mechanism at work to create the change in osmotic effect 16.

As illustrated by FIG. 7, in capillaries 2 in systemic tissue 42, the primary source of the change in osmotic effect 16 is the diffusion of HCO₃⁻ ions 26 in the capillary 2 from the region of high HCO₃⁻ ion 26 concentration at the venous end 8 of the capillary 2 toward the region of low HCO₃⁻ ion 26 concentration at the arterial end 6 of the capillary 2. Diffusion of the HCO3-ions 26 is illustrated by arrow 46 on FIG. 7.

As the HCO₃⁻ ions 26 diffuse from the venous end 8 toward the arterial end 6 of the capillary 2, the ions 26 drag on the plasma water 24 through which they diffuse 46 and alter the internal tension of the water in the plasma 24 just like the internal tension of pure liquid water is altered by lessening the pressure applied to it by 33 Torr. The internal tension of the water in the plasma 24 is altered the most at the venous end 8 of the capillary 2. The altered water in the plasma 24 pulls return fluid 11 from interstitial fluid 18 into the plasma 24 at the venous end 8 of the capillary 2 where the hydrostatic pressure 4 in plasma 24 is much less than 33 Torr. At the same time, the altered internal tension of the plasma water 24 retards the extravasation of fluid 10 from plasma 24 into the interstitial fluid 18 at the arterial end 6 of the capillary 2 where the hydrostatic pressure 4 exceeds 33 Torr. The net result is that extravasation of fluid 10 is reduced and most of the fluid 10 extravasated from plasma 24 into interstitial fluid 18 is returned as return fluid 11 to the plasma 24 at the venous end 8 of the capillary 2.

The osmotic effect 16 of diffusing bicarbonate ions 26 is also load dependent, i.e., as more CO₂ 44 is produced in active systemic tissue 42 (e.g., muscle), more HCO₃ ions 26 diffuse 46 upstream in the capillary 2 plasma 24 and have a greater osmotic effect 16 to pull return fluid 11 from interstitial fluid 18 to the plasma 24 at the venous end 8 of the capillary 2.

2. The Osmotic Force in Pulmonary Tissue.

As illustrated by FIGS. 8 and 9, in the alveoli 22 of the lung the osmotic effects of diffusing bicarbonate 26 and strong ions are reversed. Plasma 24 laden with the metabolic waste product bicarbonate (HCO₃⁻) 26 enters the arterial end 6 of the pulmonary capillary 2. The bicarbonate (HCO₃⁻) 26 leaves the plasma 24 in the form of CO₂ 44 and enters the alveoli 22 of the lung as the plasma 24 travels through the capillary 2. As a result, the HCO₃⁻ ion 26 concentration decreases as the plasma 24 flows from the arterial end 6 to the venous end 8 in the pulmonary capillaries 2. The concentrations of the strong ions Na⁺ and Cl⁻ also change so as to maintain electroneutrality. The HCO3-26, the Na+ and the Cl− ions each diffuses from its area of higher concentration to its area of lower concentration within the plasma 24 within the capillary 2. The diffusion of HCO3-ions 26 for alveolar tissue 22 is illustrated by arrow 48 of FIG. 9. The principal osmotic effect of these ions is the osmotic effect 16 created by the HCO₃⁻ ions 26 as they drag on the water in the plasma 24 through which they diffuse 48. In humans at rest, this osmotic effect 16 is about 33 Torr at the arterial end 6 of the pulmonary capillary 2, where the osmotic effect 16 retards extravasation. In humans in strenuous exercise,¹⁵ the plasma 24 osmolarity increases. However, as plasma 24 flows from end to end 6, 8 in a pulmonary capillary 2, its osmolarity may decrease as much as 27 milliosmol/liter due, in part, to a decrease in the HCO₃⁻ ion concentration from 33.2 milliosmol/liter to 23.7 milliosmol/liter, a decrease of 9.5 milliosmol/liter. At rest, this decrease is only 1.5 milliosmol/liter.

In exercise, pulmonary arterial pressure 4 increases and more fluid 10 is extravasated from plasma 24 into adjacent alveolar fluid 18 of the lung. Note again that the osmotic effect 16 of diffusing HCO3-ions 26 is load dependent in the pulmonary capillaries 2. That is, as more work is performed, more HCO₃⁻ ions 26 are formed, the ions diffuse 48 from arterial 6 to venous ends 8 of the pulmonary capillaries 2 at higher rate, and the osmotic effect 16 of the HCO₃⁻ ions 26 is as much as 184 Torr, compared with 29 Torr at rest. As a result, less fluid 10 is extravasated into the alveolar fluid 18 and more return fluid 11 is removed from the alveolar fluid 18 at a higher rate, thereby avoiding pulmonary edema.

The diffusion 48 of bicarbonate ions 26 yields the primary osmotic effect 16 for prevention of pulmonary edema. Contrary to Starling's hypothesis and equation and contrary to current accounts of the forces involved in plasma 24-interstitial fluid 18 exchange, the role of the colloid osmotic pressure of plasma proteins is minor. Furthermore, Starling's osmotic force is not load dependent; it is constant regardless of rate of work.

D. Minimizing HAPE

Since diffusion of HCO₃⁻ ions 26 creates the necessary osmotic effect 16 to retard extravasation of fluid 10 from the capillaries 2 or to return extravasated fluid 18 to the capillaries 2, an adequate rate of production of CO₂ 44 is essential for avoidance of pulmonary edema. At high altitude, inspired O₂ is insufficient to metabolize adequate carbon to CO₂ 44 and hence to HCO₃⁻ ions 26 in the plasma 24. Food ingested by high altitude climbers can supply some of the required oxygen.

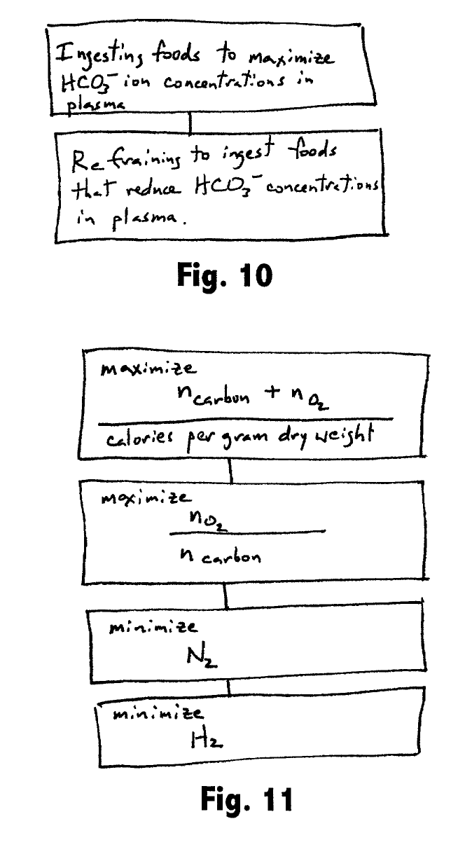

Food ingested by climbers should maximize the content of carbon and oxygen atoms (FIGS. 10, 11 ). Atoms that are metabolized to something other than CO2 should be minimized or eliminated from the diet. The food should minimize hydrogen content and should eliminate nitrogen atoms (FIG. 11). For these reasons, proteins (meat and legumes) should be eliminated from the food ingested at the highest elevations of the climb. Only digestible carbohydrates that yield the highest ratio of

$$\frac{{n}_{carbon} + {n}_{{o}_{2}}}{calories per gram dry weight}$$

and the highest ratio moles of O₂ to moles of carbon,

$$\frac{{n}_{{O}_{2}}}{{n}_{carbon}} ,$$

should be ingested (FIG. 11). Pure glucose C_{6 H}_{12 O}₆ and/or sucrose (C₁₂H₂₄O₁₂) is recommended. Glucose has sufficient oxygen to metabolize all of its hydrogen. Metabolism of the carbon requires inspired oxygen. A digestible carbohydrate with less hydrogen would be better and fats are less desirable because the ratio

$$\frac{{n}_{{O}_{2}}}{{n}_{carbon}}$$

is less favorable.

References

1) West, J. B., Mathieu-Costello, O. (1991) Stress failure in pulmonary capillaries: a mechanism for high altitude pulmonary edema. Hypoxia and Mountain Medicine. 7^{th} International Hypoxia Symposium . Ed. Sutton, J. R. and Coates, G. pp 229-39.

2) Klocke, D. L., Decker, W. W., Stepenek, J. (1998) Altitude-related illnesses. Mayo Clin. Proc. 73: 988-93.

3) Starling, E. H. (1896) On the absorption of fluids from connective tissue spaces. J. Physiol. London, 19: 312-326.

4) Michel, C. C. (1996) One hundred years of Starling's hypothesis. NIPS 11 229-237.

5) Taylor, A. E. and T. M. Moore. (1999) Capillary fluid exchange. Adv. Physiol. Ed. 22: S203-S210.

6) Hulett G. Beziehung zwischen negativem Druck und osmotischem Druck. Z. Phys. Chem. 1903; 42: 353-368.

7) Hammel, H. T. (1994) How solute alters water in aqueous solutions. J. Phys. Chem. 98: 4196-204.

8) Mysels, K. J. (1978) Solvent tension or solvent concentration? J. Chem. Ed. 55: 21-22.

9) Lewis, G. N. (1908) The osmotic pressure of concentrated solutions, and the laws of the perfect solution. J. Am. Chem. Soc. 30: 668-683.

10) Hammel, H. T. (1999) Evolving ideas about osmosis and capillary fluid exchange. FASEB J. 13: 213-31.

11) Hammel, H. T. (1995) Role of colloidal molecules in Starling's hypothesis and in returning interstitial fluid to the vasa recta. Am. J. Physiol. 268:H2133-44.

12) Hammel, H. T., Brecheu, W. F. (2000) Plasma-ISF fluid exchange in tissue is driven by diffusion of carbon dioxide and bicarbonate in presence of carbonic anhydrase. FASEB 14: Abstract 315.3.

13) Hammel, H. T. (2001) Osmotic effects on solvent of solute diffusing in solution. Advanced Research in Physical Chemistry. (In press).

14) Hammel, H. T., Brecheu, W. F. (2001) Causes of plasma-ISF exchange in fish, birds and mammals. FASEB, Abstract.

15) McKenna, M. J., Heigenhauser, G. J. F, McKelvie, R. S., MacGougal, J. D. and Jones, N. L. (1997) Sprint training enhances ionic regulation during intense exercise in man. J. Physiol. 501:687-411.

16) Porcelli, M. J., Gugelchuk, G. M. (1995) A trek to the top: a review of acute mountain sickness. J. Am. Osteopath. Assoc. 95: 718-20.

I claim:

1. A method for minimizing the incidence and effect of High Altitude Pulmonary Edema (“HAPE”) comprising the steps of:
a. Ingesting foods selected so as to maximize the change in bicarbonate concentrations in the pulmonary arterial blood plasma;
b. Refraining from ingesting said foods that reduce said bicarbonate concentration in said blood plasma.

2. The method of claim 1, said foods that maximize said change in bicarbonate concentration comprising a digestible carbohydrate selected so as to maximize a carbon content and selected so as to maximize an oxygen content per calorie per gram dry weight of said digestible carbohydrate.

3. The method of claim 2, said digestible carbohydrate further selected so as to minimize a hydrogen content of said food.

4. The method of claim 3, said digestible carbohydrate further selected so as to maximize a ratio of moles of said oxygen to moles of said carbon.

5. The method of claim 4, said digestible carbohydrate selected so as to minimize a content of fats as not maximizing the ratio of moles of said oxygen to moles of said carbon.

6. The method of claim 5, said digestible carbohydrate comprising glucose (C₆H₁₂O₆)

7. The method of claim 5, said digestible carbohydrate comprising sucrose (C₁₂H₂₄O₁₂)

8. The method of claim 1, said foods that reduce said bicarbonate concentrations further comprising said foods that contain nitrogen.

9. The method of claim 8 said foods containing nitrogen comprising meat and legumes.
